(19) United States
(12) Patent Application Publication
(10) Pub. No.: US 2026/0345526 A1
(43) Pub. Date: Nov. 5, 2026

(54) Amphenicol Adjuvant Improves Bioavailability and Efficacy

(71) Applicants: HP Ingredients Corp., Bradenton, FL (US); PHARMACEUTICAL PATENT ATTORNEYS, LLC, Miami, FL (US)

(72) Inventors: Juan Luis HANCKE OROZCO, Valdivia (CL); Annie ENG, Bradenton, FL (US)

(73) Assignee: HP Ingredients Corp., Bradenton, FL (US)

(21) Appl. No.: 19/100,148

(22) PCT Filed: Jul. 31, 2023
(86) PCT No.: PCT/US2023/071313

§ 371 Date: Jan. 31, 2025

(51) Int. Cl. A61K 31/165; A23K 20/121; A23K 20/195; A23K 50/80; A61K 31/365; A61P 31/04

(52) U.S. Cl. CPC A61K 31/165; A23K 20/121; A23K 20/195; A23K 50/80; A61K 31/365; A61P 31/04

(57) ABSTRACT

Amphenicols are a class of antibiotic. They are widely used in e.g., aquaculture. Administering an amphenicol with an andrographolide increases the bioavailability of and efficacy of amphenicols. This enables one to reduce the dose of amphenicol. This reduces the amount of residual amphenicol in the harvested animal.

### CROSS-REFERENCE TO RELATED APPLICATIONS

This is a National Stage entry of Patent Cooperation Treaty application Serial No. PCT/US2023/071313 filed 31 Jul. 2023.

### STATEMENT REGARDING FEDERALLY SPONSORED RESEARCH OR DEVELOPMENT

None.

### The Names Of The Parties To A Joint Research Agreement

The assignee contracted Salmones Antarctica S. A to test this invention.

### REFERENCE TO A SEQUENCE LISTING

None.

### STATEMENT REGARDING PRIOR DISCLOSURES BY THE INVENTOR

None.

### BACKGROUND

Amphenicols are a class of antibiotics with a phenylpropanoid structure. They function by blocking the enzyme peptidyl transferase on the 50S ribosome subunit of bacteria. Examples of amphenicols include chloramphenicol, thiamphenicol, azidamfenicol, and florfenicol. The first-in-class compound was chloramphenicol, introduced in 1949. Chloramphenicol was initially discovered as a natural product and isolated from the soil bacteria Steptomyces venezuelae. Now, however, all amphenicols are made by chemical synthesis.

Amphenicols are widely used in farming. For example, NUFLOR® florfenicol (Schering-Plough Animal Health Inc.) is used in fish farming. It is used to prevent or treat infection of the fish by Piscirickettsia salmonis. P. salmonis is the bacterial causative agent of piscirickettsiosis, an epizootic disease in salmonid fishes. It has a major impact on salmon populations, with a mortality rate of up to 90% in some species. The type strain, LF-89, is from Chile, but multiple strains exist, and some are more virulent than others. P. salmonis and piscrickettsiosis are present in various geographic regions from Europe to Oceania to South America, but the Chilean salmon farming industry has been particularly hard-hit. The disease caused by P. salmonis, piscirickettsiosis, was first identified in Chile in 1989 as “coho salmon syndrome,” although observations of the illness date to at least 1981. P. salmonis was first described in 1992, when it was identified as the causative agent of the disease. When piscirickettsiosis was first reported in 1989, it was one of the greatest threats to salmon aquaculture in Chile, with some infection coho salmon populations experiencing mortality rates of 90%.

### BRIEF DESCRIPTION OF THE INVENTION

Amphenicols such as florfenicol provide farmers a valuable tool to prevent or treat pathogenic bacterial infection by e.g., P. salmonis. Antibiotic dosing, however, poses two problems. First, large doses of antibiotics in animals that are farmed and intended to be used as food risks contaminating the animal tissue with residual antibiotic. Alternatively, small doses of antibiotic may not suffice to eliminate all the pathogenic bacteria, thus favoring the emergence of antibiotic-resistant bacteria strains. The art thus needs a way to reduce the dose of antibiotic without reducing efficacy.

We found a way. We found that andrographolide and its salts improve the bio-availability of amphenicol-class antibiotics. Using an andrographolide as an adjuvant with the antibiotic thus enables one to use a lower antibiotic dose to achieve the same efficacy.

### BRIEF DESCRIPTION OF THE DRAWINGS

FIGS. 1 and 2. Cytotoxicity of andrographolide sodium bisulfite (PubChem CID: 133555830, “AP1”) and andrographolide sulphonate (PubChem CID: 72191954, “AP2”) in combination with florfenicol in SHK-1 cells treated and infected with P. salmonis. Cells were treated with AP1 (FIG. 1) and AP2 (FIG. 2) at 4.6 and 9 ng/mL and florfenicol in the range of 0,125; 0,250; 0.5 and 1 g/mL. LDH analysis was done day 12 post-infection. Cells+Ps, cells infected without treatment; Vehicle+Ps, cells infected and treated with ethanol. “Flo sin AP” means florfenicol without an andrographolide; “Flo+AP” means florfenicol with an andrographolide. The percentage of cytotoxicity was calculated with respect to the control of total lysis of cells and the negative control (cells without treatment and infection) with the control florfenicol without AP. *p>0.001;**** p>0.001 ANOVA y Fisher.

FIG. 3. Plasma concentration of florfenicol over time: distribution of levels of plasma florfenicol. Y axis=Florfenicol plasma concentration (g/L). X axis=time (t=0, 5 days and 12 days). For each of the three days reported, the left bar is for fish treated with florfenicol alone, the right bar is for fish treated with florfenicol and also fed andrographolide. At both 5 and 12 days, fish fed andrographolide show higher average plasma florfenicol levels.

FIG. 4 measures accumulated mortality over 14 days for coho salmon treated with florfenicol alone and combined with andrographolide.

### DETAILED DESCRIPTION

Andrographolide (PubChem CID: 5318517) is a labdane diterpenoid that has been isolated from the stem and leaves of Andrographis paniculata. Andrographolide is an extremely bitter substance. Andrographolide has been studied for its effects on cell signaling, immunomodulation, and stroke. A study has shown that andrographolide may bind to a spectrum of protein targets including NF-κB and actin by covalent modification. Andrographolide has several known salts. These include andrographolide sodium bisulfite (PubChem CID: 133555830) and andrographolide sulphonate (PubChem CID: 72191954).

### Example 1

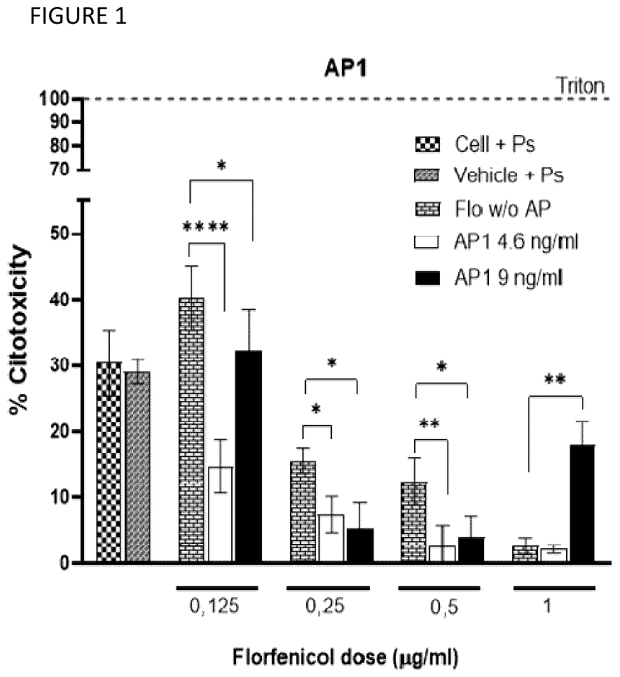

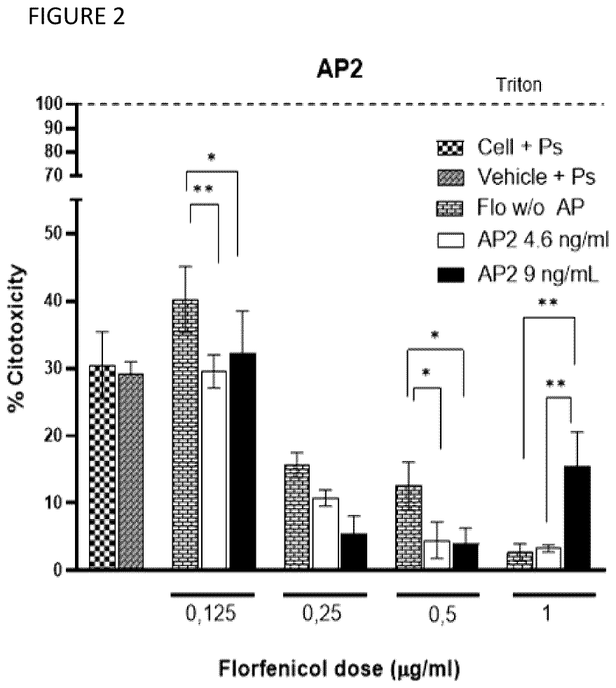

Our data in FIG. 1 shows that andrographolide sodium bisulfite and andrographolide sulphonate (AP1 and AP2, respectively) each decrease the minimum inhibitory concentration of florfenicol on P. salmonis (0.5 to 0.25 ug/mL) in infected SHK-1 cells. The effect of andrographolide on the antibiotic action of florfenicol, was corroborated by LDH in the supernatant in cells with different treatments showing the synergistic effect of andrographolide (AP) in the range of 0.125; 0.25 and 0.5 μg/mL of the antibiotic. The assay suggests a reduction of cytopathic effect of P. salmonis on SHK-1 cells when an andrographolide is combined with florfenicol.

### Example 2

Table 1 shows results of in vivo testing in coho salmon. Florfenicol (“FF”) was administered at a dose of 20 mg/kg at 15 days and at 30 days before harvesting. Andrographolide and seaweed powder were mixed with conventional fish feed at a dose of 1.056 kg seaweed powder and 0.044 kg andrographolide (“NATCONTROL”) per ton of feed. The NORMAL group corresponds to fish fed antibiotic without andrographolide.

TABLE 1
Test Report No. 44.217-22
Cage No. 101
Release Date 4 days after sampling date; UTAS = 79

| N | Diet | Weight | florfenicol |
|---|---|---|---|
| 1 | andrographolide | 2840 | <24 |
| 2 | andrographolide | 2860 | <24 |
| 3 | andrographolide | 2280 | <24 |

Test Report No. 44.218-22
Cage No. 102
Release Date 4 days after sampling date; UTAS = 79

| N | Diet | Weight | florfenicol |
|---|---|---|---|
| 1 | normal | 1880 | 270 |
| 2 | normal | 3340 | 363 |
| 3 | normal | 2270 | 502 |

Test Report No. 44.215-22
Cage No. 103
Release Date 4 days after sampling date; UTAS = 79

| N | Diet | Weight | florfenicol |
|---|---|---|---|
| 1 | andrographolide | 1970 | <24 |
| 2 | andrographolide | 3090 | <24 |
| 3 | andrographolide | 3980 | <24 |

Test Report No. 44.216-22
Cage No. 104
Release Date 4 days after sampling date; UTAS = 79

| N | Diet | Weight | florfenicol |
|---|---|---|---|
| 1 | normal | 2750 | 212 |
| 2 | normal | 2860 | 94 |
| 3 | normal | 2530 | 352 |

Table 1 surprisingly shows that andrographolide hastens the in vivo metabolism and clearance of florfenicol (“FF”). So the farmer can treat with antibiotic closer to harvest, and harvested fish have far less antibiotic residue in their meat. Table 1 also surprisingly shows that andrographolide also increases antibiotic concentration in plasma. This enables one to use a smaller dose of antibiotic to achieve the same effect. The residues of florfenicol at various days post-treatment indicate a more-rapid metabolization of florfenicol in fish muscle receiving andrographolide (<24 or not detected). These data show that andrographolide enables one to reduce the antibiotic dose.

### Example 3

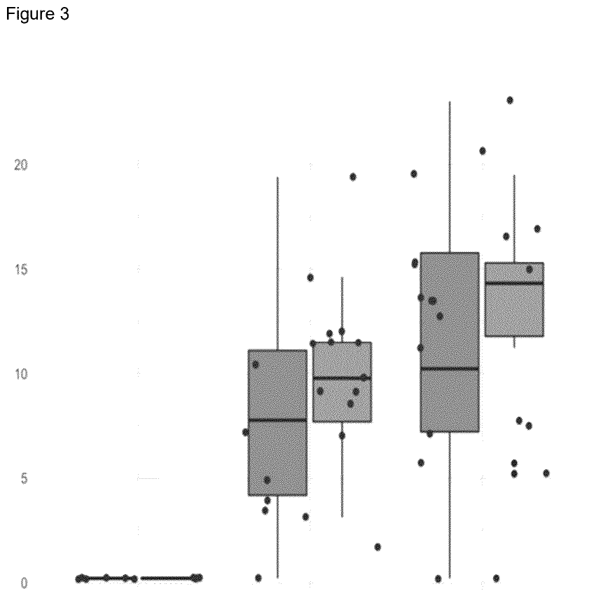

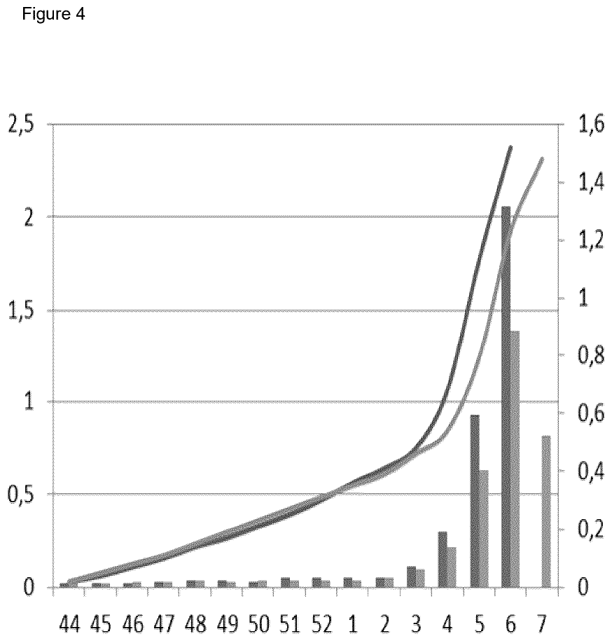

Example 3 shows results of further in vivo testing in coho salmon. Odd-numbered cages received feed medicated with both florfenicol and with andrographolide (0.044 kg andrographolide per ton of feed) for 15 days. Even-numbered cages received feed medicated with only florfenicol. Results are shown in FIG. 3. X Axis=day; Y axis (left)=accumulated mortality; Y axis (right)=periodic mortality. For each period, the left bar is florfenicol alone, the right bar is florfenicol and andrographolide. For cumulative mortality, the upper line (at date #5) is florfenicol alone, the lower line is florfenicol and andrographolide.

Andrographolide decreases mortality. In addition, it changes the causes of mortality. Table 2 compares the cause of death for the two groups.

TABLE 2
Cause / Time of Death

% of Total Deaths

| Cause | Flor | Flor + AP |
|---|---|---|
| Mechanical damage | 41.6 | 35.7 |
| SRS (skin lesions) | 25.0 | 22.5 |
| No apparent cause | 9.3 | 15.4 |
| In holding pen before harvesting | 10.9 | 0 |
| Runt (very small) fish | 0 | 11.0 |

Table 3 compares the quality of the fish at harvest time. Premium Quality (“PRM”) fish have an absence of skin lesions and adequate body weight. A total of 332,315 fish were treated with florfenicol+andrographolide (“Impares Nat Control”) and 301,334 fish were treated with florfenicol alone (“Pares Normal”). Co-administration of andrographolide increases the percentage of Premium Quality fish from 76.1% to 92.2%:

| | |
|---|---|
| Impares Nat Control | Pares Normal |

| | No | % | No | % |
|---|---|---|---|---|
| PRM | 306541 | 92.2441057 | 229190 | 76.05846 |
| GA1 | 15064 | 4.53304846 | 53835 | 17.8655578 |
| GA2 | 1620 | 0.48748928 | 4297 | 1.42599242 |
| FA | 9090 | 2.73535651 | 14012 | 4.64998971 |
| Total | 332315 | 100 | 301334 | 100 |

Premium quality is closely related to skin lesions. This study surprisingly shows that the combination significantly increases the yield of premium quality fish.

### SUMMARY

Given our disclosure, the artisan can readily derive modifications and variants. For example, our experiments use florfenicol. We expect, however, to find similar advantages with other amphenicol-class antibiotics. Similarly, our experiments used andrographolide, andrographolide sodium bisulfite and andrographolide sulphonate. We expect equivalent andrographolide salts and analogs to achieve similar results. We thus intend the legal coverage of our patent to be defined not by the specific examples we teach here, but by our legal claims and their permissible equivalents.

In the appended claims, we use the term “Andrographolide” (capitalized) to refer to this general class of compounds and “andrographolide” (lower case) to refer to the specific compound PubChem CID: 5318517.

1. A method for increasing the efficacy of an amphenicol antibiotic in fish farming, the method comprising: administering to a farmed animal fish feed containing an amphenicol antibiotic and administering fish feed containing an Andrographolide, the Andrographolide administered in an amount and for a time sufficient to decrease the amount of amphenicol antibiotic required for an antibiotic effect in said farmed fish.

2. The method of claim 1, where the amphenicol antibiotic is florfenicol.

3. The method of claim 1, where the Andrographolide is selected from the group consisting of: andrographolide, andrographolide sodium bisulfite and andrographolide sulphonate.

4. The method of claim 3, where the Andrographolide comprises andrographolide.

5. The method of claim 1, where the fish feed contains both an amphenicol antibiotic and an Andrographolide, the Andrographolide administered in an amount sufficient to decrease the amount of amphenicol antibiotic required for an antibiotic effect in said fish.

6. The method of claim 122, where the fish is salmonid.

7. The method of claim 6, where the amount of amphenicol antibiotic required for an antibiotic effect is an amount effective against Piscirickettsia salmonis.

8. A method comprising administering, to fish intended for human consumption, fish feed comprising an amphenicol antibiotic and fish feed comprising an Andrographolide, the Andrographolide administered in an amount and for a time sufficient to reduce the time the amphenicol antibiotic remains in the fish.

9. The method of claim 8, where the amphenicol antibiotic is florfenicol.

10. The method of claim 8, where the Andrographolide is selected from the group consisting of: andrographolide, andrographolide sodium bisulfite and andrographolide sulphonate.

11. The method of claim 10, where the Andrographolide comprises andrographolide.

12. The method of claim 11, where the fish feed contains both an amphenicol antibiotic and an Andrographolide, the Andrographolide administered in an amount sufficient to decrease the amount of amphenicol antibiotic required for an antibiotic effect in said fish.

13. The method of claim 24, where the fish is salmonid.

14. The method of claim 13, where the amount of amphenicol antibiotic required for an antibiotic effect is an amount effective against Piscirickettsia salmonis.

15. The method of claim 8, where the method comprises administering about 0.044 kg andrographolide per ton of feed.

16. The method of claim 12, where the method comprises administering about 0.044 kg andrographolide per ton of feed.

17. A method to farm fish, the method comprising administering to fish feed having an Andrographolide and an amphenicol antibiotic.

18. The method of claim 17 where the Andrographolide is selected from the group consisting of: andrographolide, andrographolide sodium bisulfite and andrographolide sulphonate.

19. The method of claim 18, where the Andrographolide comprises andrographolide.

20. The method of claim 19, comprising administering about 0.044 kg andrographolide per ton of fish feed.

21. The method of claim 2026, where the fish is salmonid.

22. The method of claim 1, where the fish is finned.

23. The method of claim 1, where the fish is crustacean.

24. The method of claim 8, where the fish is finned.

25. The method of claim 8, where the fish is crustacean.

26. The method of claim 17, where the fish is finned.

27. The method of claim 17, where the fish is crustacean.
